(19) United States
(12) Patent Application Publication
(10) Pub. No.: US 2026/0357173 A1
(43) Pub. Date: Nov. 12, 2026

(54) MICROFLUIDIC DEVICE AND METHOD FOR USING MICROFLUIDIC DEVICE

(71) Applicants: Tohoku Institute of Technology, Sendai-shi, Miyagi (JP); Zeon Corporation, Tokyo (JP)

(72) Inventors: Ikuro SUZUKI, Sendai-shi, Miyagi (JP); Kenichi HIROSE, Tokyo (JP); Makoto YAMANAKA, Tokyo (JP); Hiroshi KOYANAGI, Tokyo (JP); Kenji HATAKEYAMA, Tokyo (JP)

(73) Assignees: Tohoku Institute of Technology, Sendai-shi, Miyagi (JP); Zeon Corporation, Tokyo (JP)

(21) Appl. No.: 19/126,996

(22) PCT Filed: Nov. 2, 2023
(86) PCT No.: PCT/JP2023/039607

§ 371 Date: May 2, 2025

(30) Foreign Application Priority Data
Nov. 2, 2022 (JP) 2022-176317

(51) Int. Cl. C12M 3/06; C12M 1/00

(52) U.S. Cl. CPC C12M 23/16; C12M 23/34

(57) ABSTRACT

A microfluidic device includes: a first chamber capable of culturing nerve cells; and a channel extending laterally from the first chamber, the channel being capable of culturing neurites growing from the nerve cells, wherein the channel has a first end communicating with the first chamber and a second end opposite the first end, and the channel has a barrier portion at a place remote from the second end to lower a level of a part of a ceiling surface of the channel and limit entry of the nerve cells.

### TECHNICAL FIELD

The present invention relates to a microfluidic device and a method for using the microfluidic device.

### BACKGROUND ART

In order to quickly develop an agent that has an effect on a neurological disease, it is necessary to culture nerve cells and neurites (also referred to as axons) in vitro (outside a living body) and assess the efficacy of the agent on the nerve cells, the neurites, and junctions of the nerve cells and the neurites.

Conventionally, several devices made to culture nerve cells and neurites have been proposed.

Patent Document 1 and Patent Document 2 each disclose a microfluidic device having a channel capable of culturing neurites growing from cell bodies or spheroids.

### PRIOR ART DOCUMENTS

### Patent Documents

Patent Document 1: JP-B2-6430680

Patent Document 2: JP-A-2016-502921

### SUMMARY OF THE INVENTION

### Problems to be Solved by the Invention

Unfortunately, in Patent Document 1 and Patent Document 2, a cross-sectional area of the channel is relatively small to separate cell bodies and axons from each other. As a result, oxygen, carbon dioxide, and nutrients necessary for growth of the axons run short, and the growth of the axons to 1 mm or longer disadvantageously takes time.

In view of the above problems, it is an object of the present invention to provide a microfluidic device designed to efficiently separate nerve cells from neurites growing from the nerve cells in a short time and culture the nerve cells and the neurites, as well as provide a method for using such a microfluidic device.

### Means for Solving the Problems

A microfluidic device according to the present invention includes: a first chamber capable of culturing nerve cells; and a channel extending laterally from the first chamber, the channel being capable of culturing neurites growing from the nerve cells,
wherein the channel has a first end communicating with the first chamber and a second end opposite the first end, and
the channel has a barrier portion at a place remote from the second end to lower a level of a part of a ceiling surface of the channel and limit entry of the nerve cells.

According to this configuration, the barrier portion is designed to limit entry of the nerve cells. This makes it possible to separate the nerve cells cultured in the first chamber from the neurites growing from the nerve cells and being cultured in the channel. A part of the ceiling surface on a side adjacent to the second end with respect to the barrier portion is high in level. This enables satisfactory supply of oxygen and nutrients to the neurites and thus enables the neurites to grow in a short time. Consequently, the microfluidic device according to the present invention is designed to efficiently separate nerve cells from neurites growing from the nerve cells in a short time and culture the nerve cells and the neurites.

In the microfluidic device according to the present invention, the channel may have a pair of side wall surfaces to suppress growth of the neurites in a width direction of the channel. According to this configuration, the channel is designed to promote the growth of the neurites in a predetermined direction while restricting a spread of the neurites in the width direction.

In the microfluidic device according to the present invention, the pair of the side wall surfaces may extend parallel to each other. This configuration allows the neurites to grow uniformly in the width direction.

In the microfluidic device according to the present invention, a width of the barrier portion may be equal to a width of the channel at the second end. According to this configuration, the barrier portion is disposed across the width of the channel (a distance between the pair of the side wall surfaces). Thus, the barrier portion is designed to appropriately limit entry of the nerve cells.

In the microfluidic device according to the present invention, a width of the channel at the second end may be 100 μm or more and 3000 μm or less. According to this configuration, a satisfactory observation range can be obtained, and the spread of the neurites in the width direction is readily restricted.

In the microfluidic device according to the present invention, a width of the barrier portion may be 100 μm or more and 3000 μm or less. According to this configuration, a satisfactory observation range can be obtained, and the spread of the neurites in the width direction is readily restricted.

The microfluidic device according to the present invention may include a second chamber communicating with the second end of the channel. This configuration makes it possible to inject a culture medium or an agent into the channel using the second chamber.

In the microfluidic device according to the present invention, the channel may have an opening with an open top on a side adjacent to the second end with respect to the barrier portion. According to this configuration, the channel has the opening. This enables satisfactory supply of oxygen and nutrients to the neurites and thus enables the neurites to grow in a short time.

In the microfluidic device according to the present invention, a width of the barrier portion may be equal to a width of the opening. This configuration enables satisfactory supply of oxygen and nutrients to the neurites passing through the barrier portion.

In the microfluidic device according to the present invention, the channel may have a bottom surface in such a way that a height from the bottom surface to the ceiling surface at the barrier portion is 20 μm or more and 80 μm or less. According to this configuration, with the height set to 20 μm or more, the neurites are permitted to pass through the barrier portion and readily grow by expanding across the channel in the width direction. With the height set to 80 μm or less, the barrier portion effectively inhibits the entry of the nerve cells into the channel.

The microfluidic device according to the present invention may include a reservoir being disposed above the first chamber and communicating with the first chamber. This configuration enables the reservoir to store a large amount of a culture medium and enables satisfactory supply of oxygen and nutrients to the nerve cells and the neurites.

In the microfluidic device according to the present invention, a bottom surface of the first chamber and a bottom surface of the channel may be formed flush with each other. This configuration facilitates entry of the neurites growing from the nerve cells into the channel.

In the microfluidic device according to the present invention, the first chamber may have a constricted portion that is an inclining area of an inner wall at a place opposite the first end of the channel. According to this configuration, since the first chamber has the constricted portion, the nerve cells seeded in the first chamber are readily positioned.

A method for using a microfluidic device according to the present invention is a method for using the microfluidic device described above, the method including:
a first step of seeding the nerve cells in the first chamber; and
a second step of injecting, after the first step, a culture medium into the first chamber and the channel,
wherein, in the second step, the culture medium is injected to reach a level that is above the part of the ceiling surface at the barrier portion and below a part of the ceiling surface not at the barrier portion.

Another method for using a microfluidic device according to the present invention is a method for using the microfluidic device described above, the method including:
a first step of seeding the nerve cells in the first chamber; and
a second step of injecting, after the first step, a culture medium into the first chamber and the channel,
wherein the opening is located below an upper end of the first chamber, and
wherein, in the second step, the culture medium is injected to reach a level that is above the opening and below the upper end of the first chamber.

The method for using the microfluidic device according to the present invention is designed to efficiently separate nerve cells from neurites growing from the nerve cells in a short time and culture the nerve cells and the neurites.

### BRIEF DESCRIPTION OF THE DRAWINGS

FIG. 1 is a plan view of a microfluidic device according to a first embodiment.

FIG. 2 is a sectional view of the microfluidic device taken along a line II-II shown in FIG. 1.

FIG. 3 is a schematic sectional view showing a state in which the microfluidic device according to the first embodiment is used.

FIG. 4 is a plan view of a microfluidic device according to a second embodiment.

FIG. 5 is a sectional view of the microfluidic device taken along a line V-V shown in FIG. 4.

FIG. 6 is a schematic sectional view showing a state in which the microfluidic device according to the second embodiment is used.

FIG. 7 is a schematic sectional view showing a state in which a microfluidic device according to a third embodiment is used.

FIG. 8 is a plan view of a microfluidic device according to a fourth embodiment.

FIG. 9 is a sectional view of the microfluidic device taken along a line IX-IX shown in FIG. 8.

FIG. 10 is a schematic sectional view showing a state in which a microfluidic device according to a fifth embodiment is used.

FIG. 11 is a sectional view of a microfluidic device according to a sixth embodiment.

### MODE FOR CARRYING OUT THE INVENTION

A microfluidic device and a method for using the microfluidic device according to the present invention will be described with reference to the drawings. It should be noted that the drawings disclosed herein merely show schematic illustrations. That is, the dimensional ratios on the drawings do not necessarily reflect the actual dimensional ratios, and the dimensional ratios are not necessarily the same between the drawings.

### First Embodiment

### <Structure of Microfluidic Device>

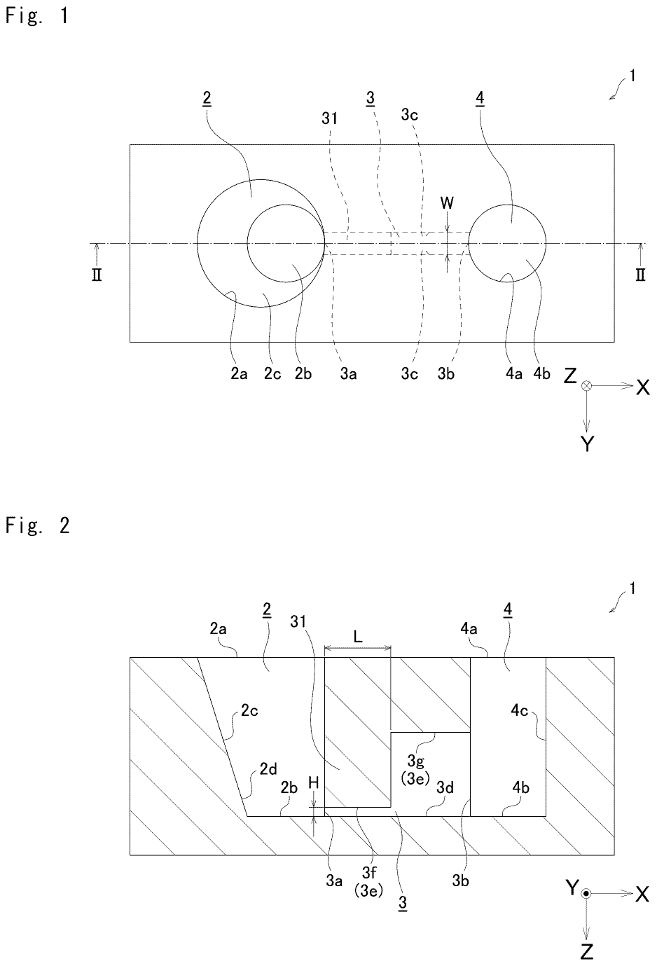

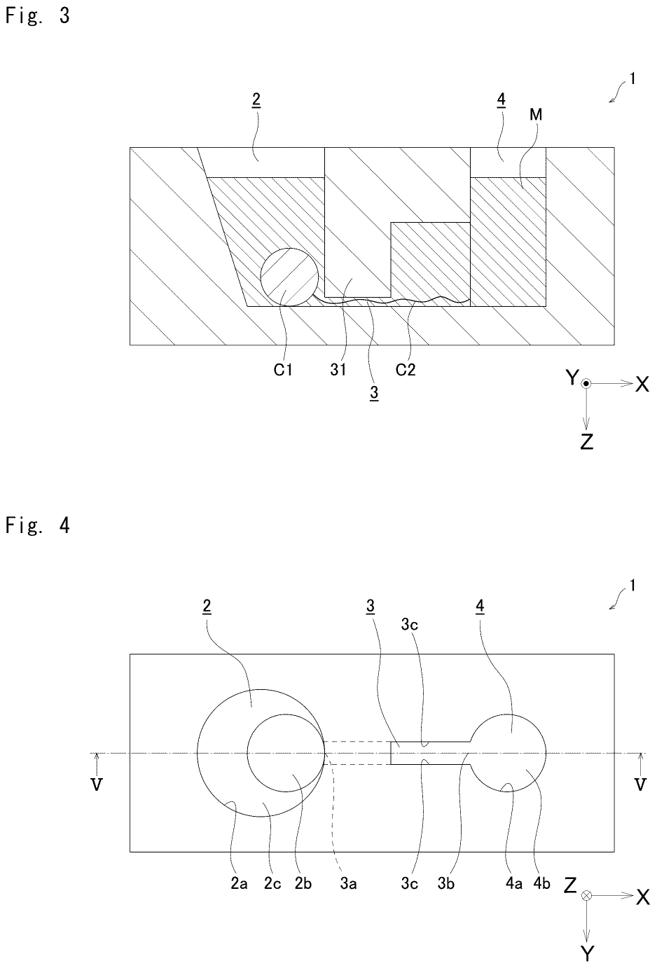

FIG. 1 is a plan view of a microfluidic device 1 according to a first embodiment. FIG. 2 is a sectional view of the microfluidic device 1 taken along a line II-II shown in FIG. 1. FIG. 3 is a schematic sectional view showing a state in which the microfluidic device 1 according to the first embodiment is used.

When it is necessary to make a distinction between positive and negative to express a direction herein, the direction is described with a positive or negative sign, such as “+X direction” or “−X direction”. When it is not necessary to make a distinction between positive and negative to express a direction, the direction is simply described as “X direction”. That is, when the direction is simply described as “X direction” herein, both “+X direction” and “−X direction” are included. The same applies to the Y direction and the Z direction. It should be noted that the microfluidic device 1 is usually used in such a manner that the Z direction corresponds to a vertical direction, and the −Z direction corresponds to an upward direction.

The microfluidic device 1 has a rectangular parallelepiped shape as a whole. The microfluidic device 1 includes a first chamber 2 and a channel 3 extending from the first chamber 2 in an X direction. In addition, the microfluidic device 1 may include a second chamber 4.

The first chamber 2 is a space used to culture nerve cells C1 shown in FIG. 3. The channel 3 is a space used to culture projecting neurites C2 growing from the nerve cells C1. In the present invention, the neurite C2 means an axon covered by myelin (a myelin sheath). The second chamber 4 is a space used mainly to store a culture medium M.

The first chamber 2 is a recess in a shape of a substantially truncated cone, and has an open top and a closed bottom. The first chamber 2 has a circular opening 2a, a circular bottom surface 2b, and an inner wall 2c connecting the opening 2a to the bottom surface 2b.

A diameter of the bottom surface 2b is smaller than a diameter of the opening 2a. Thus, the first chamber 2 has a constricted portion 2d that is an inclining area of the inner wall 2c at a place opposite a first end 3a (described later) of the channel 3. Since the first chamber 2 has the constricted portion 2d, the nerve cells C1 seeded in the first chamber 2 are readily positioned in a vicinity of the channel 3.

The diameter of the opening 2a is, for example, 500 μm or more and 10000 μm or less. The diameter of the bottom surface 2b is, for example, 100 μm or more and 500 μm or less. With the opening 2a being greater than the bottom surface 2b, work for inputting the nerve cells C1 is facilitated. Since the diameter of the bottom surface 2b is in this range, the nerve cells C1 are readily positioned.

In the present embodiment, the first chamber 2 is a recess in the shape of a substantially truncated cone, but is not limited thereto. The first chamber 2 may be a recess in a shape of a substantially quadrangular pyramid, for example. In this case, one side of a rectangular bottom surface of the first chamber 2 is, for example, 100 μm or more and 500 μm or less.

A center of the bottom surface 2b is disposed nearer to the channel 3 than a center of the opening 2a. This allows the nerve cells C1 to come nearer to the channel 3, and thus facilitates entry of the neurites C2 growing from the nerve cells C1 into the channel 3.

The second chamber 4 is a recess in a shape of a cylinder, and has an open top and a closed bottom. The second chamber 4 has a circular opening 4a, a circular bottom surface 4b, and an inner wall 4c connecting the opening 4a to the bottom surface 4b. A diameter of each of the opening 4a and the bottom surface 4b is, for example, 500 μm or more and 10000 μm or less. In like manner with the first chamber 2, the second chamber 4 may be a recess in a shape of a substantially truncated cone or a substantially quadrangular pyramid.

The channel 3 has the first end 3a communicating with the first chamber 2 and a second end 3b communicating with the second chamber 4. The first end 3a of the channel 3 is connected to a lower end of the inner wall 2c of the first chamber 2. The second end 3b of the channel 3 is connected to a lower end of the inner wall 4c of the second chamber 4.

As shown in FIG. 1, the channel 3 has a pair of side wall surfaces 3c, 3c. With the pair of the side wall surfaces 3c, 3c, the channel 3 is designed to suppress growth of the neurites C2 in a width direction (a Y direction). Hence, the channel 3 is designed to promote the growth of the neurites C2 in the X direction while restricting a spread of the neurites C2 in the width direction. The side wall surfaces 3c extend along the X direction. Preferably, the pair of the side wall surfaces 3c, 3c extend parallel to each other.

A distance between the pair of the side wall surfaces 3c, 3c, in other words, a width W of the channel 3 (refer to FIG. 1), is preferably 100 μm or more and 3000 μm or less, and more preferably 400 μm or more and 1200 μm or less. With the width W set to 100 μm or more, a satisfactory observation range can be obtained. Meanwhile, with the width W set to 3000 μm or less, the spread of the neurites C2 in the width direction (the Y direction) is readily restricted.

Preferably, widths (sizes in the Y direction) of the first chamber 2 and the second chamber 4 are greater than the width W of the channel 3. This helps to inject a culture medium M or an agent into the first chamber 2 and the second chamber 4.

As shown in FIG. 2, the channel 3 has a bottom surface 3d and a ceiling surface 3e. The bottom surface 3d extends along the X direction. The bottom surface 3d of the channel 3 is formed flush with the bottom surface 2b of the first chamber 2. This facilitates entry of the neurites C2 growing from the nerve cells C1 into the channel 3. However, the bottom surface 3d of the channel 3 does not necessarily need to be formed flush with the bottom surface 2b of the first chamber 2. The bottom surface 3d of the channel 3 and the bottom surface 2b of the first chamber 2 may differ in level.

The channel 3 has a barrier portion 31 at a place remote from the second end 3b to lower a level of a part of the ceiling surface 3e. A part of the channel 3 where the barrier portion 31 is formed has a flat sectional shape. The barrier portion 31 limits entry of the nerve cells C1 seeded in the first chamber 2 into the channel 3. This enables only the neurites C2 to grow in the channel 3.

In the present embodiment, the barrier portion 31 is disposed at the first end 3a of the channel 3, but is not limited thereto. The barrier portion 31 may be disposed at a place remote from the first end 3a.

The ceiling surface 3e includes a low ceiling surface 3f that is a lower surface of the barrier portion 31 and a high ceiling surface 3g that is higher than the low ceiling surface 3f from the bottom surface 3d. In other words, the ceiling surface 3e is formed in a shape of stairs.

A height H from the bottom surface 3d to the low ceiling surface 3f is preferably 20 μm or more and 80 μm or less, and more preferably 30 μm or more and 60 μm or less. With the height H set to 20 μm or more, the neurites C2 are permitted to pass through the barrier portion 31 and readily grow by expanding across the channel 3 in the width direction (the Y direction). Meanwhile, with the height H set to 80 μm or less, the barrier portion effectively inhibits the entry of the nerve cells C1 into the channel 3.

A length L of the barrier portion 31 (a length of the low ceiling surface 3f) is preferably 1 mm or less, and more preferably 0.2 mm or less. With the length L of the barrier portion 31 set to 1 mm or less, the growth of the neurites C2 is boosted in a direction of extension of the channel 3 (in the X direction) and is allowed to come close to original growth of the neurites C2.

The channel 3 includes the high ceiling surface 3g on a side adjacent to the second end 3b with respect to the barrier portion 31. This enables satisfactory supply of oxygen and nutrients to the neurites C2 and thus enables the neurites C2 to grow in a short time.

The microfluidic device 1 is made of a transparent thermoplastic resin. Examples of the transparent thermoplastic resin include polystyrene (PS), polymethyl methacrylate (PMMA), cycloolefin polymer (COP), and cycloolefin copolymer (COC). The thermoplastic resin that the microfluidic device 1 is made of is particularly preferably medical-grade COP.

As described above, the microfluidic device 1 of the present embodiment includes the first chamber 2 capable of culturing the nerve cells C1 and the channel 3 extending laterally (in the X direction in the present embodiment) from the first chamber 2, the channel 3 being capable of culturing the neurites C2 growing from the nerve cells C1. The channel 3 has the first end 3a communicating with the first chamber 2 and the second end 3b opposite the first end 3a. The channel 3 has the barrier portion 31 at a place remote from the second end 3b to lower the level of a part of the ceiling surface 3e of the channel 3 and limit entry of the nerve cells C1. This configuration makes it possible to culture the nerve cells C1 and the neurites C2 that are efficiently separated from each other by the barrier portion 31. Moreover, a part of the ceiling surface 3e on the side adjacent to the second end 3b with respect to the barrier portion 31 is high in level. This enables satisfactory supply of oxygen and nutrients to the neurites C2 and thus enables the neurites C2 to grow in a short time.

### <Method for Using Microfluidic Device>

Hereinbelow, a method for using the microfluidic device 1 will be described. Specifically, the method is a method for culturing nerve cells C1 using the microfluidic device 1.

First, in a first step, nerve cells C1 are seeded in the first chamber 2 from the opening 2a. The nerve cells C1 in spheroids or organoids are seeded. The nerve cells C1 are not limited to spheroids of nerve cells and organoids of nerve cells, but may be a culture medium in which nerve cells are dispersed. Next, in a second step, a culture medium M is injected into the first chamber 2 and the channel 3. The culture medium M is also injected into the second chamber 4. In an example shown in FIG. 3, the culture medium M is injected to reach a level above the ceiling surface 3e of the channel 3. The nerve cells C1 are cultured in the first chamber 2 in this state for several days, and thus neurites C2 grow from the nerve cells C1 and enter into the channel 3. At this time, the barrier portion 31 limits entry of the nerve cells C1. This makes it possible to culture the nerve cells C1 and the neurites C2 that are separated from each other. The neurites C2 cultured in the channel 3 can be observed from outside. The neurites C2 are observed, for example, from a lower side (a +Z direction side) of the microfluidic device 1 using an instrument such as a fluorescent microscope. Since the culture medium M in the channel 3 is in contact with the ceiling surface 3e at this time, observability is not compromised by a meniscus of a liquid surface of the culture medium M. Seeding described herein refers to a concept including a case in which a culture medium is put in the first chamber 2 and the channel 3 that are empty and then a culture medium containing spheroids, organoids, and/or nerve cells is put therein, and a case in which a culture medium containing nerve cells and/or the like is directly put in the empty first chamber 2.

After that, an agent can be administered from the opening 4a of the second chamber 4, for example, to assess efficacy and/or toxicity of the agent on the neurites C2. As described above, the microfluidic device 1 according to the present invention makes it possible to perform a series of operations from culturing to agent testing and observation and assessment in a single device.

While the embodiment of the present invention has been described above with reference to the drawings, it should be construed that specific configurations are not limited to the above embodiment. The scope of the present invention is indicated not only by the above description of the embodiment but also by the claims, and all changes which come within the meaning and range of equivalency of the claims are therefore intended to be embraced therein.

The structure adopted in each embodiment described above can be adopted in any other embodiment. Specific configurations of parts are not restricted only to those in the above-described embodiment, and various modifications are possible within the scope of the gist of the present invention.

### Second Embodiment

A second embodiment of a microfluidic device according to the present invention will be described by mainly focusing on points different from the first embodiment. It should be noted that components common to those of the first embodiment will be denoted by the same reference numerals, and description thereof will be omitted as appropriate.

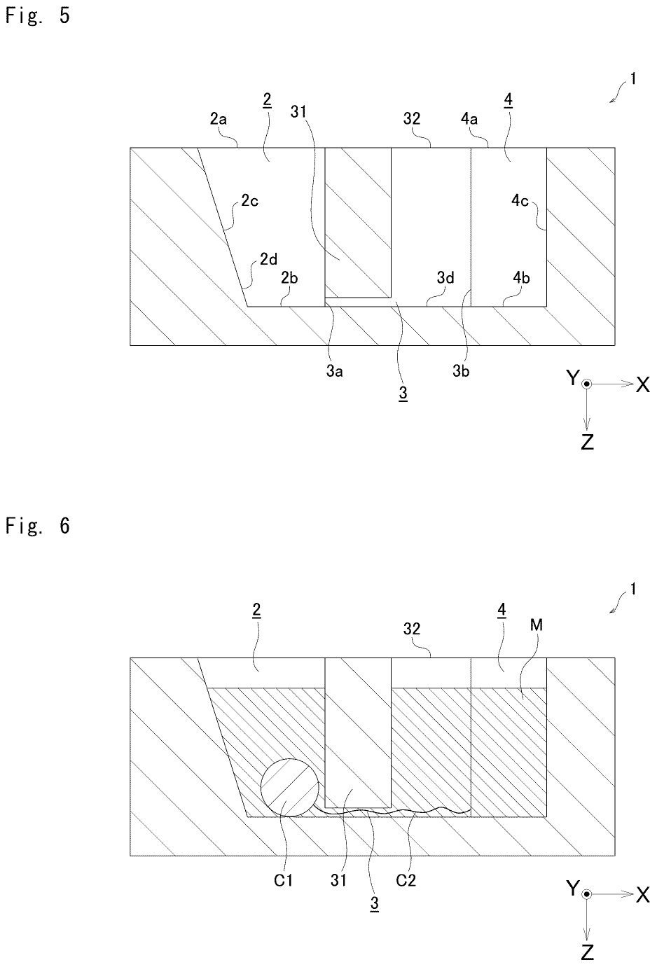

FIG. 4 is a plan view of a microfluidic device 1 according to a second embodiment. FIG. 5 is a sectional view of the microfluidic device 1 taken along a line V-V shown in FIG. 4. FIG. 6 is a schematic sectional view showing a state in which the microfluidic device 1 according to the second embodiment is used. In the microfluidic device 1, a channel 3 may have an opening 32 with an open top on a side adjacent to a second end 3b with respect to a barrier portion 31. In other words, a ceiling surface of the channel 3 may have the opening 32. In this example, the high ceiling surface 3g of the first embodiment does not exist, and the channel 3 includes an open top by the opening 32. The channel 3 has the opening 32. This enables, as shown in FIG. 6, satisfactory supply of oxygen and nutrients to neurites C2 and thus enables the neurites C2 to grow in a short time.

### Third Embodiment

A third embodiment of a microfluidic device according to the present invention will be described by mainly focusing on points different from the first embodiment. It should be noted that components common to those of the first embodiment will be denoted by the same reference numerals, and description thereof will be omitted as appropriate.

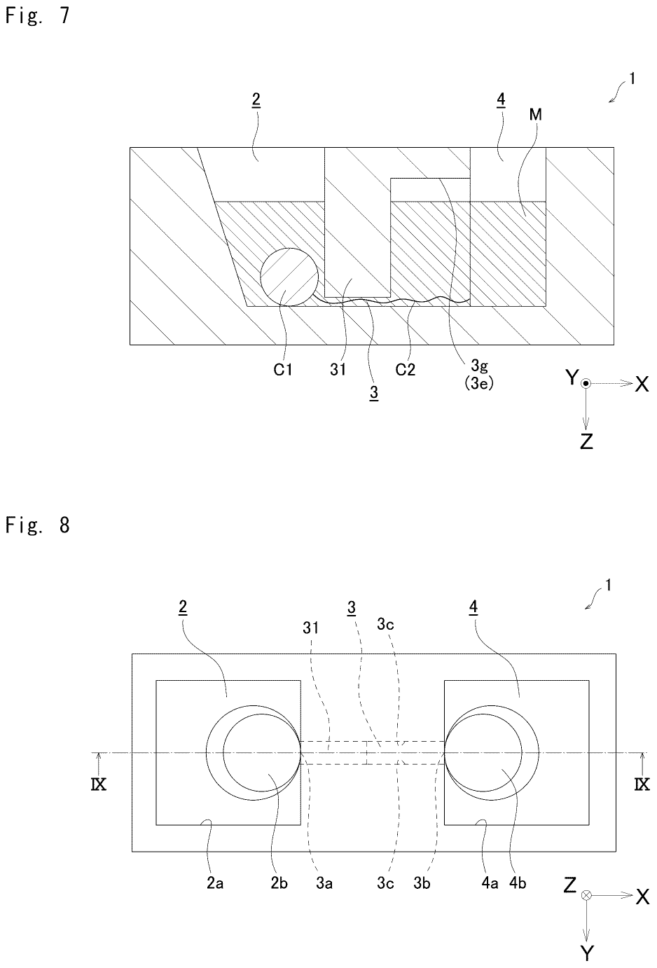

FIG. 7 is a schematic sectional view showing a state in which a microfluidic device 1 according to the third embodiment is used. In the third embodiment, a high ceiling surface 3g of a channel 3 is higher in level than that in the first embodiment. In the second step, a culture medium M is injected to reach a level that is above a part of a ceiling surface 3e (a low ceiling surface 3f) at a barrier portion 31 and below a part of the ceiling surface 3e (the high ceiling surface 3g) not at the barrier portion 31.

In a similar way to the first and the second embodiments, this enables the microfluidic device 1 according to the third embodiment to satisfactorily supply oxygen and nutrients to neurites C2 and thus enable the neurites C2 to grow in a short time. The microfluidic device 1 according to the third embodiment is designed to suppress evaporation of the culture medium M and enhance strength of the channel 3 compared to the second embodiment in which the channel 3 has the opening 32.

### Fourth Embodiment

A fourth embodiment of a microfluidic device according to the present invention will be described by mainly focusing on points different from the first embodiment. It should be noted that components common to those of the first embodiment will be denoted by the same reference numerals, and description thereof will be omitted as appropriate.

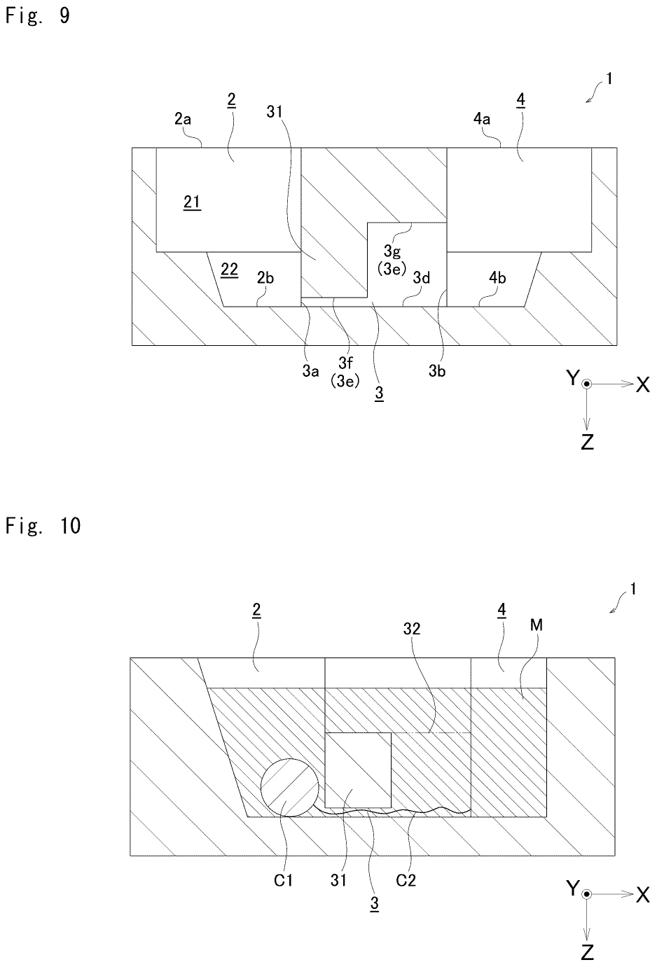

FIG. 8 is a plan view of a microfluidic device 1 according to the fourth embodiment. FIG. 9 is a sectional view of the microfluidic device 1 taken along a line IX-IX shown in FIG. 8. A first chamber 2 of the fourth embodiment includes an upper part 21 in a shape of a rectangular parallelepiped and a lower part 22 in a shape of a substantially truncated cone. An opening 2a differs in shape from a bottom surface 2b, and the opening 2a has a rectangular shape. As shown in FIG. 8, the upper part 21 of the first chamber 2 is wider than the lower part 22 of the first chamber 2 in plan view. This enables the upper part 21 to function as a reservoir and enables the first chamber 2 to store a large amount of a culture medium M in the upper part 21. A second chamber 4 is substantially similar in shape to the first chamber 2. This also enables the second chamber 4 to store a large amount of the culture medium M.

### Fifth Embodiment

A fifth embodiment of a microfluidic device according to the present invention will be described by mainly focusing on points different from the first embodiment. It should be noted that components common to those of the first embodiment will be denoted by the same reference numerals, and description thereof will be omitted as appropriate.

FIG. 10 is a schematic sectional view showing a state in which a microfluidic device 1 according to the fifth embodiment is used. In the fifth embodiment, an opening 32 of a channel 3 is located below an upper end of a first chamber 2. The opening 32 is level with an upper end of a barrier portion 31, and it can be said that the upper end of the barrier portion 31 is located below the upper end of the first chamber 2.

In the second step in the fifth embodiment, a culture medium M is injected to reach a level that is above the opening 32 and below the upper end of the first chamber 2. Hence, an upper part of the first chamber 2 communicates with the channel 3 through the opening 32. As a result, a growth factor for neurites C2 generated from nerve cells C1 in the first chamber 2 is readily mixed with the culture medium M in the channel 3 through the opening 32. This makes it possible to encourage growth of the neurites C2.

### Sixth Embodiment

A sixth embodiment of a microfluidic device according to the present invention will be described by mainly focusing on points different from the first embodiment. It should be noted that components common to those of the first embodiment will be denoted by the same reference numerals, and description thereof will be omitted as appropriate.

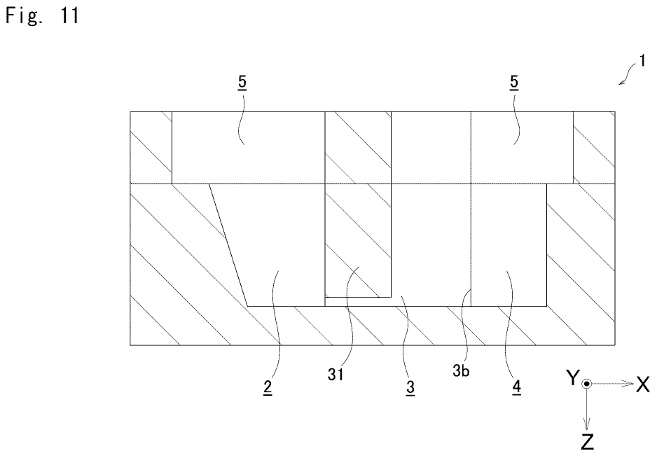

FIG. 11 is a sectional view of a microfluidic device 1 according to the sixth embodiment. The microfluidic device 1 includes a reservoir 5 being disposed above a first chamber 2 and communicating with the first chamber 2. The microfluidic device 1 may also include a reservoir 5 being disposed above a second chamber 4 and communicating with the second chamber 4. The reservoir 5 is designed to store a large amount of a culture medium M.

In FIG. 11, the reservoirs 5 are shown as components separate from the first and the second chambers 2 and 4 respectively, but may be integrated with the respective chambers. In other words, the reservoirs may be parts of the first and the second chambers 2 and 4, respectively. For instance, in the fourth embodiment shown in FIGS. 8 and 9, the upper part 21 of the first chamber 2 functions as a reservoir.

### DESCRIPTION OF REFERENCE SIGNS

1 Microfluidic device
2 First chamber
2a Opening of first chamber
2b Bottom surface of first chamber
2c Inner wall of first chamber
2d Constricted portion
3 Channel
3a First end of channel
3b Second end of channel
3c Side wall surface of channel
3d Bottom surface of channel
3e Ceiling surface of channel
3f Low ceiling surface
3g High ceiling surface
4 Second chamber
4a Opening of second chamber
4b Bottom surface of second chamber
4c Inner wall of second chamber
5 Reservoir
21 Upper part of first chamber
22 Lower part of first chamber
31 Barrier portion
32 Opening
C1 Nerve cell
C2 Neurite
H Height from bottom surface of channel to low ceiling surface
W Width of channel
L Length of barrier portion
M Culture medium

1. A microfluidic device comprising:
a first chamber capable of culturing nerve cells; and
a channel extending laterally from the first chamber, the channel being capable of culturing neurites growing from the nerve cells,
wherein the channel has a first end communicating with the first chamber and a second end opposite the first end, and
the channel has a barrier portion at a place remote from the second end to lower a level of a part of a ceiling surface of the channel and limit entry of the nerve cells.

2. The microfluidic device according to claim 1, wherein the channel has a pair of side wall surfaces to suppress growth of the neurites in a width direction of the channel.

3. The microfluidic device according to claim 2, wherein the pair of the side wall surfaces extend parallel to each other.

4. The microfluidic device according to claim 3, wherein a width of the barrier portion is equal to a width of the channel at the second end.

5. The microfluidic device according to claim 1, wherein a width of the channel at the second end is 100 μm or more and 3000 μm or less.

6. The microfluidic device according to claim 1, wherein a width of the barrier portion is 100 μm or more and 3000 μm or less.

7. The microfluidic device according to claim 1, comprising a second chamber communicating with the second end of the channel.

8. The microfluidic device according to claim 1, wherein the channel has an opening with an open top on a side adjacent to the second end with respect to the barrier portion.

9. The microfluidic device according to claim 8, wherein a width of the barrier portion is equal to a width of the opening.

10. The microfluidic device according to claim 1, wherein the channel has a bottom surface in such a way that a height from the bottom surface to the ceiling surface at the barrier portion is 20 μm or more and 80 μm or less.

11. The microfluidic device according to claim 1, comprising a reservoir being disposed above the first chamber and communicating with the first chamber.

12. The microfluidic device according to claim 1, wherein a bottom surface of the first chamber and a bottom surface of the channel are formed flush with each other.

13. The microfluidic device according to claim 1, wherein the first chamber has a constricted portion that is an inclining area of an inner wall at a place opposite the first end of the channel.

14. A method for using the microfluidic device according to claim 1, the method comprising:
a first step of seeding the nerve cells in the first chamber; and
a second step of injecting, after the first step, a culture medium into the first chamber and the channel,
wherein, in the second step, the culture medium is injected to reach a level that is above the part of the ceiling surface at the barrier portion and below a part of the ceiling surface not at the barrier portion.

15. A method for using the microfluidic device according to claim 8, the method comprising:
a first step of seeding the nerve cells in the first chamber; and
a second step of injecting, after the first step, a culture medium into the first chamber and the channel,
wherein the opening is located below an upper end of the first chamber, and
wherein, in the second step, the culture medium is injected to reach a level that is above the opening and below the upper end of the first chamber.
